(19) United States
(12) Patent Application Publication
(10) Pub. No.: US 2023/0176015 A1
(43) Pub. Date: Jun. 8, 2023

(54) ADVANCED CALIPER FOR A PIPE AND METHOD OF USE

(71) Applicant: King Fahd University of Petroleum and Minerals, Dhahran (SA)

(72) Inventors: Rakan FADHEL, Dhahran (SA); Salaheldin ELKATATNY, Dhahran (SA)

(73) Assignee: King Fahd University of Petroleum and Minerals, Dhahran (SA)

(21) Appl. No.: 17/543,221

(22) Filed: Dec. 6, 2021

(51) Int. Cl. G01N 29/12; G01B 5/12; G01P 15/125; G01P 15/09; G01P 1/00; G01C 19/5712; G01N 27/82; G01N 29/24; G01N 33/2045

(52) U.S. Cl. CPC G01N 29/12; G01B 5/12; G01P 15/125; G01P 15/09; G01P 1/00; G01C 19/5712; G01N 27/82; G01N 29/2437; G01N 33/2045; G01N 2291/0234; G01N 2291/0289; G01N 2291/101; F16L 2101/30

(57) ABSTRACT

A robotic device and method for inspecting a pipeline to assess metal loss, the presence of defects and corrosion effects. The robotic device is an inline inspection tool that can establish a positional address in the pipeline using known positional benchmarks. The robotic device comprises flexible electronic caliper sensors measuring pipe diameter and an elastic foam body to prevent seizing within the pipeline. A removable PCB enables interchangeable operation with in-kind devices of different diameters and/or with the computers, extracting and plotting the data. The method of measurement may use data fusion between different instruments and measurement methodologies.

### BACKGROUND OF THE INVENTION

### Technical Field

The present disclosure relates to a method, system and apparatus for evaluating the inner surface of a pipe including identifying and characterizing the presence of corrosion and defects on the wall of the inner surface of the pipe as well as the positional characterization of the defects.

### Description of the Related Art

The “background” description provided herein is for the purpose of generally presenting the context of the disclosure. Work of the presently named inventors, to the extent it is described in this background section, as well as aspects of the description which may not otherwise qualify as prior art at the time of filing, are neither expressly or impliedly admitted as prior art against the present disclosure.

Hydrocarbon-carrying pipes are exposed to highly corrosive environments, due to the combined presence of acidity and moisture in crude oil and gas. This acidity is naturally derived (e.g., mainly organic and inorganic sulfurous and acidic compounds in crude petroleum and hydrogen sulfide of natural gas) or introduced by stimulation operations such as acid injection. Other components of the corrosive environment include high concentrations of electrolytes which can function to facilitate galvanic defect-promoting currents between sites in the pipe with different tension or microstructure. Other components are elemental sulfur, polysulfides and sulfides, and the products of oxidation of organic and inorganic materials in air.

Development of hidden defects in pipe walls is especially problematic, weakening the structure and elevating the risk of the development of cracks and ruptures under increased pressure. Such increased pressures are necessary for transporting both petroleum and natural gas to the pipeline destinations.

Transmission pipelines move hydrocarbon products from production regions to distribution centers and operate at pressures ranging from 200 up to 1,200 psi, with each transmission line using compressor stations (for gas lines) and pump stations (for crude oil and liquid products) (see: Pipeline Accident Report: Rupture of Hazardous Liquid Pipeline With Release and Ignition of Propane, Carmichael, Miss., Nov. 1, 2007; Washington, D.C.: National Transportation Safety Board. 2009; Pipeline Accident Report: Pipeline Rupture and Subsequent Fire in Bellingham, Wash., Jun. 10, 1999, Washington, D.C.: National Transportation Safety Board. 2002; Pipeline Accident Report: Natural Gas Pipeline Rupture and Fire Near Carlsbad, N. Mex., Aug. 19, 2000, Washington, D.C.: National Transportation Safety Board. 2003). Local heating that accompanies the release of pressurized gas or accidental fire due to the presence of electric devices or sparks creates safety problems alongside product loss. The resulting safety consequences are even more severe in refinery and chemical plant operations where the concentration of combustible materials in a small area makes each of these sites extremely dangerous. The investment and the economic significance of these installations requires even more stringent control than during the transportation stage. Thus, the internal inspection of the pipelines is an inseparable component of the hydrocarbon transporting and processing cycle.

Inspections of transport facilities such as pipelines are often conducted by “pig” devices, with the term originating due to the typical appearance of the devices. In-line inspection (ILI) “smart pigs” travel through pipelines helping perform analysis and preventative maintenance before an incident can occur. Since 1999, corrosion caused pipeline incidents are down 76% with the help of ILI smart pigs (see AOPL data, incorporated herein by reference).

Smart pigs detect metal loss and wall cracks as small as 1 mm deep and 25 mm long with a 90% probability of detection. While no technology is error-proof, the ability of ILI “pigs” to detect minute defects long before they are a threat to the pipeline provides an advantage over other inspection techniques such as hydrostatic pressure testing (using water at high pressures inside a pipe to test pipe integrity at that point in time). Pipeline operators use ILI smart pigs, to inspect pipelines by traveling through and scanning the pipe walls. This is accomplished by inserting the “pig” into a “pig launcher” (or “launching station”)— an oversized section in the pipeline, reducing to the normal diameter. The launching station is then closed, and the pressure-driven flow of the product in the pipeline propels the pig until it reaches the receiving trap—the “pig catcher” (or “receiving station”). The operation is risky, especially in pressurized lines.

ILI smart pigs produce large amounts of raw data which must be analyzed to separate the natural features of the pipe metal from the potential problems. The raw data can be displayed graphically or in 3D to help operators determine the severity of a potential problem. Pipeline operators use analytical models to predict the growth rate of a corrosion area or crack so they can schedule maintenance before the issue threatens the pipe's integrity.

ILI smart pigs also called “tools” by pipeline operators, are grouped into three main categories according to the potential problem they are designed to find.

Dents—Dent smart pigs, also called deformation or geometry tools, use flexible calipers to measure a pipe's shape. Dent tools also find buckles, wrinkles or other types of bending strain that may indicate pressure on or movement of the pipe walls.

Corrosion tools—Corrosion smart pigs primarily use magnetic fields that detect metal loss in a pipe, which can indicate general corrosion, pitting, pinholes or wall thinning from erosion (internal wearing away of the pipe). Technical names for corrosion tool types include MFL (magnetic flux leakage) and TFI (transverse field inspection) tools.

Crack—Crack tools use ultrasonic waves, or specialized magnetic or analytical approaches, to find cracks or defects in the pipe wall, connecting welds or dents. Technical names for the types of crack tools include UT (ultrasonic testing) and TFI (transverse field inspection) tools.

Preferably, ILI is carried out in a manner that is reliable and integrated with pipeline operations. A single launch of the pig device is preferably used to check for all sources of pipe damage. Preferably, the “pig” device is positionally oriented with high precision, such that it reports the longitudinal position of the damage site relative to the launch station and also the angular position of the damage on the wall, as well as prioritize the damages by severity. The tools capable of all roles are not widespread.

Di Lullo et al. in “A novel fully plastic caliper pig for low-risk pipeline inspection—Design, characterization and field test” available at https:_www.researchgate.net/publication/301399775_A_novel_fully_plastic_caliper_pig_for_low-risk_pipeline_inspection_-_Design_Characterization_and_Field_Test disclose a caliper sensing system with flexible arms, with the input signals originating from strain gauges embedded in the caliper arms and from a three-axial accelerometer integrated on a PCB to determine the pig orientation along the pipe. Other parts include a 2 GB mini-SD card memory slot for data storage, an auxiliary SDRAM for temporary data storage, the main microcontroller for measurement and system management, a dedicated microcontroller for USB interface control, a real-time clock and the battery charger circuits. The resulting device is described as a single-channel Caliper pig, equipped with a mechanical measuring system designed to sense the internal pipe diameter, an odometer and an internal locator unit. This element consists of a short-range locator designed to find the pig position in case of a pig-stuck event. The foam caliper pig assigns each detected defect to a specific pipe sector, thus distinguishing localized defects from concentric welds. Lullo et al. does not mention GPS-free orientation system.

US20190346333 to Youcef-Toumi et al. discloses systems and methods for localizing a robot in a water pipe system or other fluid conduit based in part on obtrusions detected by the robot travelling through the system. The obtrusions can include pipe joints that connect consecutive standard fixed-length pipe segments. By detecting such repeating obtrusions, the robot can estimate its speed and/or the relative distance that it has travelled within the pipe system. The robot can be configured to localize itself by detecting consecutive pipe joints and other obtrusions in the pipe system using on-board tactile sensors. Tactile sensors can be configured to stretch or compress as they contact the obtrusions along the inner wall of a pipe system. The device can be configured to provide acceleration measurements that are indicative of changes or variation in the robot's speed. The device can also be used to measure and/or provide directional data that is indicative of a direction that the robot is heading. Youcef-Toumi measure timing by integrating the acceleration profile and not directly by a clock. Yocef-Toumi et al. discloses retractable arms connected to positional sensors that allow measuring the inner diameter of the piping. These arms, however, are not bent backwards and are retractable, not flexible and not plastic.

U.S. Pat. No. 6,243,657B1 to Tuck et al. discloses a pipeline inspection and defect mapping system that includes a pig having an inertial measurement unit and a pipeline inspection unit for recording pig location and defect detection events, each record time-stamped. The system also includes several magloggers (magnetic logging units) at precisely known locations along the pipeline, each containing a fluxgate magnetometer for detecting the passage of the pig along the pipeline and further containing a clock synchronized with the clock in the pig. The locations of the various magloggers are known in a north/east/down coordinate system through a differential global positioning satellite process. Finally, a postprocessing off-line computer system receives downloaded maglogger, inertial measurement, and odometer data and using Kalman filters, derives the location of the detected defects in the north/east/down coordinate frame.

Notwithstanding these features of conventional pig systems in use for ILI, there is a need for a simple, inertially-oriented ILI “pigging” device which is GPS-independent, capable of recognizing metal-loss defects as well as corrosion and crack defects and preferably operates independently from an external navigation aids such as magloggers.

Accordingly, it is one object of the present disclosure to provide an ILI device and system that identifies the positional and angular addresses of defects of all kinds, prognosticates the propagation of the damage, prioritizes remediation, is simple, non-redundant and self-propelling.

### SUMMARY OF THE INVENTION

According to a first aspect, the invention relates to an internal caliper with electric sensors having higher accuracy as compared with mechanical sensors connected to the retractable/flexible measuring legs.

According to a second aspect, a caliper is modular, and comprises a removable circuit module and retractable/bendable sensor arms, wherein the removable circuit enables the user to work for different pipe diameters, wherein the working diameters can be gauged by using different arms of different lengths.

According to a third aspect, the sensor arms of the caliper are deformable and mounted on an ILI device capable of traveling through a pipe. The ILI device is preferably a pig type device having one or more foam components in contact with the pipe wall, e.g., is a foam pig.

According to a fourth aspect, the caliper contains flex sensors which enable recording changes in diameter (3D position, distance and time) of a pipe undergoing ILI, wherein the sampling frequency is adjustable (different distance between the reported measurements).

According to a sixths aspect, the device yields highly accurate three dimensions (XYZ) mapping data to support the pipeline integrity program.

According to a seventh aspect, the device and the accompanying software allow GPS-free navigation.

According to an eighth aspect, the device exchanges shareware with other ILI devices and can benefit from the experience of positional error prediction and resets in multiple systems, shared and communicated remotely.

According to a ninth aspect, the device utilizes a limited number of sensors, is economical, amenable to local production and has long battery life.

### BRIEF DESCRIPTION OF THE DRAWINGS

A more complete appreciation of the disclosure and many of the attendant advantages thereof will be readily obtained as the same becomes better understood by reference to the following detailed description when considered in connection with the accompanying drawings, wherein:

FIG. 1 shows an assembled robotic inline inspection (“pig”) foam device.

FIG. 2A shows a lateral cross-section and the components of a foam “smart pig” device.

FIG. 2B shows the cross-section II and the components.

FIG. 2C shows the optional central hollow plastic pole 13, housing additional sensors.

FIG. 2D shows the embodiment with the two sets of calipers, front and rear.

FIG. 3 shows a circuit design for embedment in an advanced caliper for surface pipe analysis, or placement in a “pig” body.

FIG. 4 shows the detailed caliper arms design.

FIG. 5 shows a caliper arm design with an optional steel strand embedded in foam.

FIG. 6 shows the wires (25) connecting caliper sensing arms and internal strain gauges (24).

FIG. 7 shows a conventional smart pig device having rigid and seizure-prone caliper arms.

FIG. 8a shows the scheme of a uniaxial capacitance micro-accelerometer.

FIG. 8b shows the scheme of a triaxial piezoresistive micro-accelerometer.

FIG. 9 shows the scheme of a conventional gimbal and rotor-based gyroscope.

FIG. 10 shows the scheme of the derivation of the Coriolis effect.

FIG. 11 shows the degrees of freedom in rotational motion (yaw, roll and pitch).

### DETAILED DESCRIPTION OF THE EMBODIMENTS

Embodiments of the present disclosure will now be described more fully hereinafter with reference to the accompanying drawings, in which some, but not all of the embodiments of the disclosure are shown.

The present disclosure will be better understood with reference to the following definitions.

As used herein, the words “a” and “an” and the like carry the meaning of “one or more”. Additionally, within the description of this disclosure, where a numerical limit or range is stated, the endpoints are included unless stated otherwise. Also, all values and subranges within a numerical limit or range are specifically included as if explicitly written out.

As used herein, the term “optional” or “optionally” means that the subsequently described event(s) can or cannot occur or the subsequently described component(s) may or may not be present (e.g. 0 wt %).

As used herein, the term “pigging” refers to the practice of using devices known as pigs or scrapers to perform various maintenance and/or inspection operations preferably without stopping the flow of the product in a pipeline.

As used herein, the term “ILI” refers to “In-line Inspection device” and the term “ILI device” indicates “ILI Pig devices”, also synonymous to “smart pig”, “intelligent pig”, “pig device”, “foam pig” in the context of this disclosure.

As used herein, the term “intelligent pig”, “smart pig” refers to pigs that include instruments that include electronics and sensors that collect various forms of data while traveling through a pipeline. The electronics are preferably sealed to prevent leakage of the pipeline product into the electronics. Many pigs use specific materials according to the product in the pipeline. Power for the electronics is typically provided by onboard batteries which are also sealed. Data recording may be by various means ranging from analog, digital, or solid-state.

As used herein, the term “artificial intelligence” (AI) refers to the simulation of human intelligence in machines that are programmed to mimic human actions. The term may also be applied to any machine that exhibits traits associated with a human mind such as learning and problem-solving.

As used herein, the term “self-programming” refers to AI that studies code posted on code-presenting platforms and uses or to write its own code (e.g., the software called Bayou or the analogous solutions). Using a process called neural sketch learning, the AI reads all the code and then associates an “intent” behind each. When a human asks Bayou to create an application, Bayou associates the intent it learned from other code and begins writing applications to address the intended purpose.

As used herein, the term “data fusion” refers to the process of integrating multiple data sources to produce more consistent, accurate, and useful information than that provided by any individual data source. Data fusion processes are often categorized as low, intermediate, or high, depending on the processing stage at which fusion takes place. Low-level data fusion combines several sources of raw data to produce new raw data. The expectation is that fused data is more informative and synthetic than the original inputs.

As used herein, the term “PCB” means “printed circuit board”, comprising electronic and controller elements of a device. The PCB is preferably insulated and connected with sensors in a removable module.

As used herein, the term “POM Plug” or “POM disk” means polyoxymethylene protective disk, e.g., a component of a pig that insulates a PCB of the “smart pig” device from the harsh working environment.

As used herein, the term “SDRAM” means “synchronous DRAM” and is a generic name for various kinds of dynamic random-access memory (DRAM) that are synchronized with the clock speed that the microprocessor is optimized for. This tends to increase the number of instructions that the processor can perform in a given time.

As used herein, the term “SD-card” means “Secure Digital”, abbreviated as SD, a proprietary non-volatile memory card format developed by the SD Card Association (SDA) for use in portable devices. Examples include modern microcontrollers have built-in SPI logic that can interface to an SD card operating in its SPI mode, providing non-volatile storage.

As used herein, the term “SPI logic” means The Serial Peripheral Interface (SPI), synchronous serial communication interface specification used for short-distance communication, primarily in embedded systems. The interface is a de-facto standard. Typical applications include Secure Digital cards.

As used herein, the term “EMAT” refers to “electromagnetic acoustic transducer”.

As used herein, the term “MFL” refers to magnetic flux leakage (TFI or Transverse Field Inspection technology), a magnetic method of nondestructive testing that is used to detect corrosion and pitting in steel structures, most commonly pipelines and storage tanks.

As used herein, the term “ART” refers to acoustic resonance technology. ART exploits the phenomenon of half-wave resonance, whereby a suitably excited resonant target (such as a pipeline wall) exhibits longitudinal resonances at certain frequencies characteristic of the target's thickness. By knowing the speed of sound in the target material, the half-wave resonant frequencies can be used to calculate the target's thickness.

As used herein, the term “IMU” refers to “inertial measurement unit” consisting minimally of a gyroscope (measuring Yew, Pitch and Roll) and an accelerometer, measuring translational acceleration.

As used herein, the term “comprehensive calibrating device” refers to an ILI pig device having a caliper, IMU, EMAT, EMF, ART, or other acoustic sensor and capable of detecting cracks, pits, washouts, ovalizations and other defects of the pipelines more effectively than a simple ILI device enabled with the caliper and IMU sensors only.

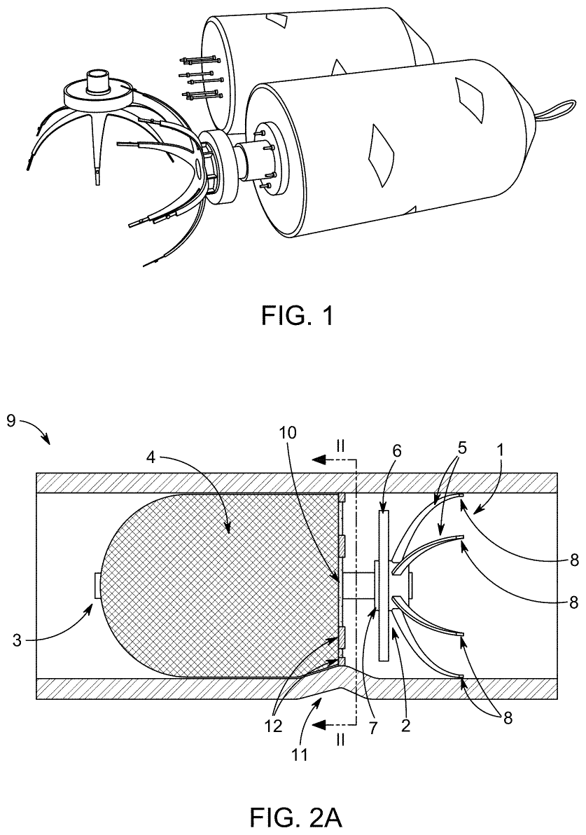

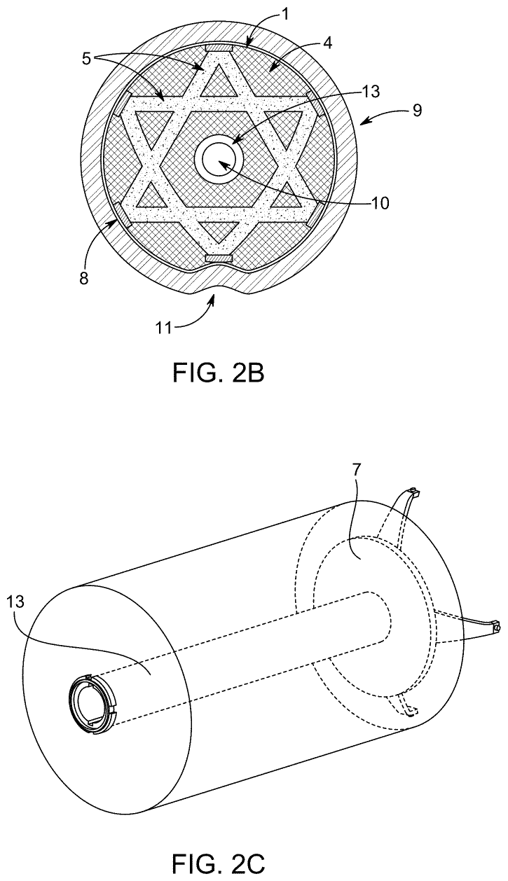

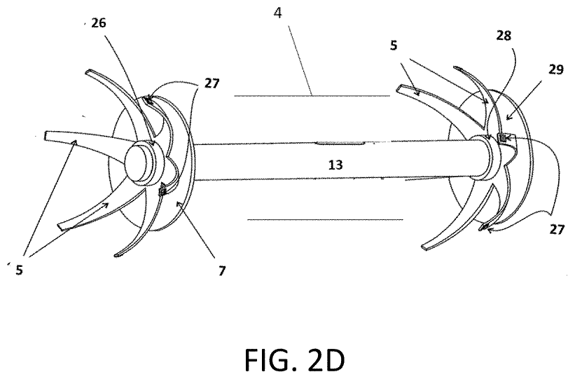

FIG. 1 presents an outlook of the disclosed ILI caliper device. FIGS. 2A and 2B are a schematic view of an apparatus according to the present invention comprising a first crown of petals (e.g., flexible arm electronic calipers with flexible caliper sensors) and a foam pig shown as it passes inside a pipeline in a sectional view. The sliding device is a foam pig 1 consisting of polymeric or expanded elastomeric material 4 perforated in the center, installed on the central body 3 in the front with respect to the first crown of petals 5. Said foam pig 4 is connected to said central body 3 by connection means (not shown) and has a perforated cylindrical form or a perforated bullet form. By installing a foam pig 4, the apparatus not only allows analysis of the pipeline and fluid contained therein but also removes possible liquid or solid deposits present in the pipeline walls. As the foam pig is made of a polymeric or expanded elastomeric material, it becomes compressed in the presence of restrictions, continuously adapting itself to the internal form of the pipeline 9. In a particular embodiment of the present invention, said foam pig 4 comprises deformation caliper sensors 5 installed therein. In particular, said deformation sensors 5 of the foam pig 4 can be flexible strip-like condensers, consisting of a polymeric-type material optionally having interior thin non-planar layers of metallic material which increase their capacity when the condenser extends. Said deformation caliper sensors 5 are installed on the foam pig 4 in pre-extended mode and fixed to it by supports 12 of plastic material which allows to keep the deformation sensors 5 in tension when the foam pig 4 is not compressed. Said deformation caliper sensors 5 are installed on the flat rear wall of the foam pig 4 so as not to superimpose the central hole of the foam pig 4. There are at least two deformation sensors 5, preferably six, in a star arrangement (FIG. 2B). In this way, possible compressions of the foam pig 4, due for example to sudden contraction 11 of the internal diameter of the pipeline 9, can be revealed and measured by said deformation sensors 5. In a particular embodiment of the present invention, said central body 13 can comprise a shutter (not shown), positioned inside the calibrated hole 10 in a transversal position (FIG. 2B, 2C).

The processor, sensor and memory components are assembled in a PCB enclosure unit 6 (compartment) of FIG. 2A that is removably connected to the central body. The data acquisition and storage module are preferably enclosed within a polyoxymethylene (POM) or rubber disc 6 to maximize its robustness and reliability which may be connected to a flange (e.g., an aluminum disc) 7 by means of a POM flange drilled with 6 through holes matching the nuts of the flange 7. The outer diameter of flange and disc together is 40-540 mm in diameter for most of the pipelines. A POM ring spacer may be added in order to improve the assembling flexibility, as it allows for the use of modules with different thickness and/or dimensions (between 6 and 7, not shown).

The PCB enclosure 6 incorporates other elements of the device, e.g., such as one or more of those described below in more detail. The system comprises a processor unit embodied on a circuit board that contains a real-time clock module, an accelerometer, a gyroscope, a memory module, a flex sensor module comprising 4 or more flex sensors, an odometer, a chargeable battery module and a battery holder.

In an embodiment of FIG. 2C, the “smart pig” device comprises one or more additional components sealed from the aggressive environment either in the thermoplastic disk or in a hollow support such as that described. In case of housing any of an EMAT, MFL, REV or ART sensors (below), the hollow support of 13 of FIG. 2A (shown in FIG. 2C as well) is preferably made of epoxy/acrylate composite and is insulated from the environment similar to the PCB unit. The plastic walls make the unit permeable to the magnetic and electromagnetic fields while providing the necessary protection.

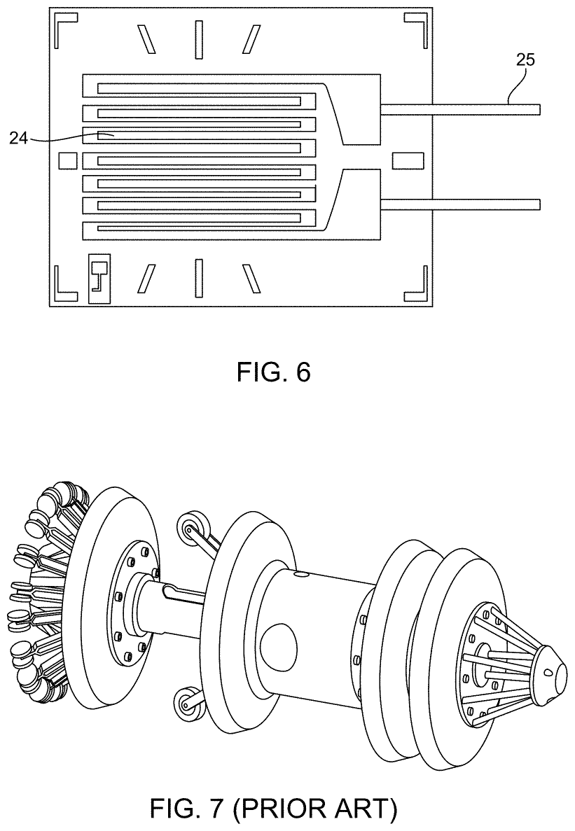

In an embodiment shown in FIG. 2D two sets of flexible calipers are mounted on the central body 13. The rear set is mounted at a stern section of the device and is described above, with the position 26 indicating the protective plug insulating the caliper central point, the PCB disk 6 and the juncture of the PCB with the flange 7 against the pipeline fluid from the rear side. All seams in the assembly can be sealed by an acrylate polymer or epoxide. The position 27 indicates flexural sensors built in the caliper and communicating with the PCB via the wire 25 (FIG. 6) passing through the flexible arms.

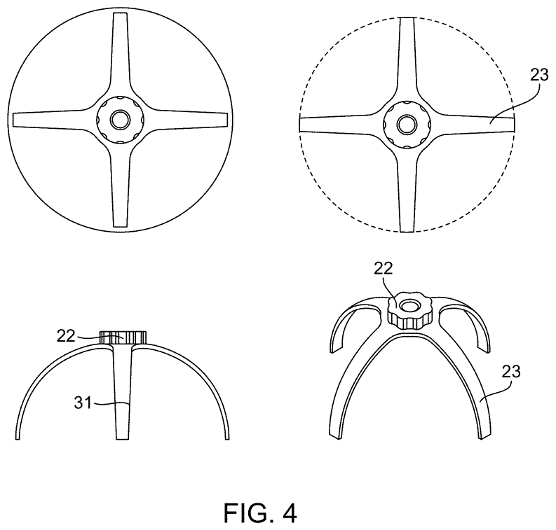

The frontal set of flexible calipers are located at a bow section of the device and are preferably inserted in the central body 13 via a cylindrical fitting 31 (FIG. 4), with the inner space of 13 tightly preferably sealed against the pipeline fluid by acrylate or epoxide placed in the juncture between the collar 28 and the outer wall of 13. The foam body 4 is sandwiched between the front and rear caliper sets. Similar flexural built-in sensors 27 can be placed on the frontal caliper set, with the electric link to PCB 6 extending via the internal space of 13. The shield 29 with the diameter equal or exceeding that of the foam body 4 serves the purpose of breaking soft deposits and gels in front of the device, to prevent false positive signaling by the front calipers, unrelated to the state of metal piping. The benefit of installing two pairs of calipers on the same device is the ability to collect additional information. The probability of identifying a defect is improved and results may be cross-validated therefore the probability of false positives by the rear set decreases.

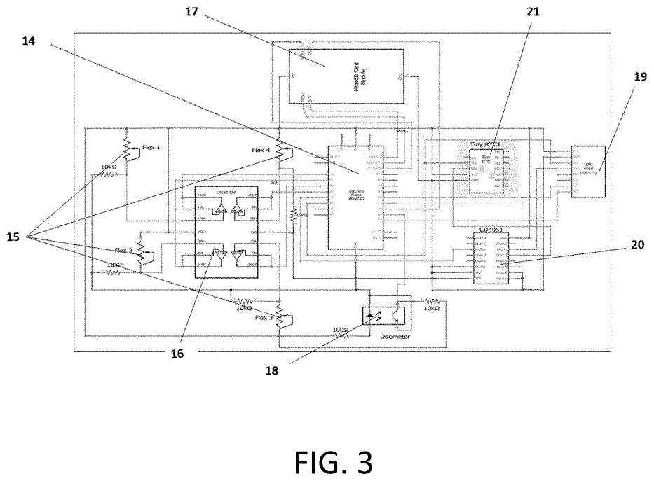

FIG. 3 shows the electronic scheme of the assembly. The position 14 is a processor, coordinating the on-board sensors, timing and recording. The positions 15 are the variable impedance circuits of the flexural caliper sensors 5 and the position 16 is an analog processing unit converting the impedance differentials into logic in communication with the central processor. The position 17 is a memory unit in communication with 14 and other subsystems, such as electronic timer 21, inertial navigation unit 19 comprising an accelerometer and a gyroscope, and a multiplexer 20 also known as a data selector, is a device that selects between several analog or digital input signals and forwards it to a single output line.

In operation, the assembly is launched through a special compartmentalized port with pressure management. The port can be a “piggable valve” (see for example U.S. Pat. No. 6,925,671, WO06128546, DE102010024871—each incorporated by reference) or a pig launching station (see for example GB1388426, U.S. Pat. Nos. 4,135,949, 9,797,541, 6,925,671, JP11138118, US2002170599, DE4010855, EP0266097—each incorporated by reference), with different embodiments incorporated herein by references in their entirety. The injected apparatus travels in the stream of the material filling the pipeline (hydrocarbon, organic solvents and reactants, water and water-based solutions).

The pig device is advantageously modular and interchangeable between the different diameters of piping. Preferably the pig device includes a removable circuit 6 to allow operation and subsequent training of other devices using similar algorithms and approaches.

The data acquisition and storage module (position 5 in FIG. 2a, position 17 in FIG. 3), is preferably a multilayer printed circuit board (PCB), which is enclosed within a thermoplastic material such as, without limiting, polyoxymethylene (POM or Delrin, Du Pont) in the form of a disc or other circular shape of this profile to maximize its robustness and reliability. The data module is preferably insertable into the foam body through a flange 7 in FIG. 2A. To allow data downloading and post-processing and battery recharging, the PCB is equipped with a computer interface (e.g., USB) protected by an airtight polymer plug. The PCB is fully coated with a specifically selected epoxy resin, in order to guarantee perfect isolation from the external environment. An important feature relates to the mechanical connection between the contacts of strain gauges which may be mounted on or emerge from the top plate of the flexible arm sensors/caliper (below), and the PCB. The PCB input wires protrude and pass through pinholes made in the bottom face of the POM disc (FIG. 2A, position 6, the right side of the disk 6, the input wires are shown in FIG. 6 describing flexural sensor below). Once welded to the strain gauge contacts they are tightened by rotation through a coupling between the threaded flange and caliper and kept in place by grooves milled on the back of the POM disc. In order to maintain modularity the entire assembly 5-8 in FIG. 2A preferable can be transferred to another pig device with another diameter of foam component or for the connection with a laptop, i.e., a feature absent in conventional devices (FIG. 7).

General principles of proper “smart pig” functioning are preferably as follows. One purpose of the device is to identify problematic sites within a piping system, preferably while the piping system is filled with the working fluid. Piping system, in the context of the present disclosure means the pipelines that carry oil, gas, wastes and related products to and from drilling, processing, distribution and manufacturing areas under high pressure. There are four main types of pipelines: gas pipelines, oil and hazardous liquid pipelines, gathering pipelines, distribution pipelines, usually made of steel. They range from just a 2-4 inches to over 40 inches in diameter. The working fluid flow provides the energy that permits the “pig” device to travel through the interior space defined by the pipes in the piping system. The device is intended to identify, record and report the positional and angular addresses of the problematic sites.

The pig device preferably operates with a data acquisition scheme and data storage procedure that maximizes sampling frequency and provides minimum power consumption. Considering that common flow velocities during pigging missions are in the range of 0.2 to 3 m/s, a reasonable minimum mission duration of 20 h can be assumed, which enables inspection of 20 to 100 km of pipelines based on the distances coverable during the battery lifetime at the above-mentioned flow velocities. The system may be configured to automatically alternate between two acquisition modes: a normal mode where all inputs are acquired at 1 kSa/s and a fast mode where only a pair of strain gauges 5, placed on opposite arms, are acquired at 16 kSa/s, while acquisition from other inputs is suspended. The time-sharing scheme is approximately 99% normal mode and 1% fast mode per second of acquisition and the arm pair selected for fast acquisition changes clockwise continuously. This solution provides periodic windows with measurements of high spectral quality, while still assuring an acceptable battery life. These higher-accuracy measurements provide sampling steps in the order of tens of micrometers (at the typical pig speed), which are suitable for fine defects detection and surface roughness estimation. A magnetic switch allows to completely shut-down the onboard electronics during long-term inactivity, to achieve a “deep sleep” battery life of up to 5 years.

In a variation of this embodiment, the sampling frequency is defined by encountering defects. After coming across a defect, the sampling frequency is increased in proportion to the size or nature of the defect. If the additional defects do not appear within a fixed section of the path, the sampling frequency decreases. Conversely, the additional problematic spots cause the increase of sampling frequency. The approach permits to cover longer sections of the tested pipeline at the same battery life.

Foam Component

The foam component (foam vector) of the “smart pig” device is shown in FIG. 1 and as 4 in FIG. 2. The foam component is an elongated cylindrical body with the aspect ratio from 1:2 to 1:5 measured as the diameter to length ratio. The front end of the foam vector (bow section opposite to the calipers) can be hemispherical, semi-elliptic, bullet-shaped or obtuse, avoiding sharp angles for improved streamlining. The foam material is configured to compress upon encountering a restriction within the bore of the pipe, i.e. the material is capable of deforming without breaking. This is advantageous in that the risk of the main instrument being damaged due to restrictions within the bore of the pipe is reduced.

Another aspect of the device is the ability of recovery from “stuck while running” situations. These eventualities are preventable by forming the body of the “pig” device from a deformable foam to help preclude the seizing of the device in the turns of the pipes.

The preferred materials suitable for providing a compressible, elastic, durable, stretchable, chemically inert and non-swelling body are expanded polyurethanes.

According to an embodiment of the present invention the expanded polyurethane has a density in a range of 30-125 kg/m³. In a preferred embodiment, the foam component is bilayer, with the outer shell having the expanded density of 30-125 kg/m³ and a more elastic inner core includes polyurethane of a density in a range of 20-60 kg/m³. Such bilayer structure is advantageous, providing enough insulation and protection by the outer layer and enough elasticity by the central core. The total thickness of the outer and inner layers is preferably in a range or ratios of 0.1:10 to 10:0.1; preferably 1:4 to 1:1 or 1:2 to 1:1.

In an exemplary and non-limiting embodiment the foam composition of the present invention is a combination of a specific polyester polyol, 1,4-butanediol, blowing agents and catalysts with a clear, medium-viscosity, modified diphenylmethane diisocyanate containing a high percentage of pure diphenylmethane diisocyanate and a lesser amount of diphenylmethane diisocyanate adducts. The preferred foam can be processed by use of a high pressure reaction injection mold which is equipped with a high pressure impingement mixing device as well as other conventional process methods, such as hand mix or ordinary dispensing mold for manufacturing or molding the foam pig with a density of as low as 2.5 pounds per cubic feet. In one non-limiting example embodiment the formulation uses 44±3 weight percent of pure adducts of diphenylmethane diisocyante, 44±3 weight percent of the polyester polyol which must be the nominal molecular weight of 450-1000 and of viscosity in the range of 120-500 centipoise at 140 degrees Fahrenheit measured by Brookfield LVF viscometer, 1.33 weight percent of Freon-113, 0.063 weight percent of catalysts, and remaining balance of colorants and pigments.

Flexible Arm Electronic Calipers

The term “calipers” refers to the flexural petal-shaped extensions in position 5 of FIG. 2a. The calipers 5 are extended outwardly contacting the inner surface of the piping system. The changes in the caliper orientation produce the changes in the torsional strain in the associated sensors, which translates in the change of Ohmic impedance. The unbalanced voltage bridge produces an analogue signal to the processor.

Preferably, the flexible caliper includes from 2 to 8, even more preferably 4 caliper arms equally radially distributed around a central point of the pig device. The arms are shown as reference numeral 5 in FIGS. 2a and 2b, implied in the description of the flexural sensors in FIG. 3 and the more detailed view is given in FIG. 4. Preferably only 4 flexible arms are included, each flexible arm representing a quadrant of measurement and analysis within a pipeline. When viewed from above, the calipers have a maximum extension that is preferably greater than the inner diameter of the pipe for which they are intended for use (FIG. 4). The maximum extension of the caliper arms can vary from 100 mm to 1220 mm (for crude petroleum), following the diameters of industrial piping, and the provided range may be broader for the chemical plants (40-1500 mm), including cylindrical reactors and tanks.

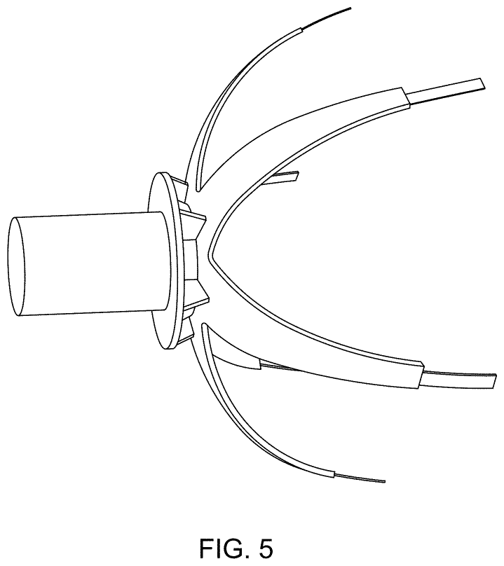

The flexible sensors are preferably embedded on upper or lower surfaces of each of the flexible arms (27, FIG. 2d) and include embodiments in which a wire pattern is printed on an upper or lower surface of the flexible arms that is sensitive to a degree of bending of the flexible arm depending on changes in the resistivity of the flux sensor embedded thereon. Alternatively, metallic resilient strands or cores are buried in flexible foamed plastic, providing the necessary linearity between the extent of flexural bending strain and the size of the irregularities on the pipeline wall. The metal strands coated by plastic foam are shown in FIG. 5.

The inventive Flex sensors are shown in FIG. 6. The sensors comprise variable interdigitated resistors 24 whose resistance varies with the amount of bend/displacement, while the sensors are incorporated into a voltage divider or bridge circuit (FIG. 3, 15) via the wires 25 to produce the corresponding voltage values. The deformation of the caliper arms pulls the wires 25 and the sensors of FIG. 6 and FIG. 3 respond with the proportional bridge unbalancing. These voltage values are acquired by the microcontroller 14 and are converted to displacement in millimeters. However, these displacement values still need to be calibrated according to the actual size and orientation of the physical arrangement. Non-limiting representative examples of such sensors include those described by Saggio et al. in Resistive flex sensors: a survey. Smart Materials and Structures. 2015 Dec. 2;25 (1):013001; Sajid M, Dang H W, Na K H, Choi K H. Highly stable flex sensors fabricated through mass production roll-to-roll micro-gravure printing system. Sensors and Actuators A: Physical. 2015 Dec. 1; 236:73-81; and U.S. patent Nos. U.S. Pat. Nos. 7,458,289; and 8,820,143; each incorporated herein by reference in its entirety).

In another embodiment, the bending of the caliper arms is detected by means of foil strain gauges embedded within the arms themselves in correspondence of the locations of maximum strain. This kind of strain gauge, in which conductive patterns are deposited on a flexible polyimide sheet, provide very high accuracy measurements and work at high temperatures of up to 180° C. Within the selected deformation range, the expected relationship between arms bending and strain gauge response can be approximated with a linear function. The pipe diameter variations can thus be obtained by simply multiplying the acquired signal by the sensitivity coefficient, k. This coefficient depends on the combination of arm geometry, material flexibility, strain gauges sensitivity, and adopted signal amplifiers gain. The gauges are protected from the pipeline fluid by placement within the arms and in this embodiment the position 25 indicates an electronic connection

The sensitivity coefficient (in the order of 100 LSB/cm) is used in order to achieve a measurement resolution of about 10 mm, required to observe the pipe roughness. This multi-channel caliper structure allows for independent arm bending, which is necessary to distinguish the different causes of diameter changes via data post-processing, thus discriminating structural elements from defects, as well as to determine the sector position and extent of asymmetrical dents or bumps. The flexible arms are preferably fabricated by vacuum casting technique.

Strain gauge sensors and related wire connectors are preferably cemented with a cyanoacrylate glue to the concave surface of each metal stripe and then buried in the polyurethane resin to provide mechanical and chemical shielding (for the protected juncture between the caliper sensor unit and the PCB disk 6 see FIG. 2a). In order to guarantee optimal contact between the caliper arms and the pipe, stainless steel preferably used if the metal strands are incorporated in the caliper arms. The smooth and rounded steel contact surface prevents jamming and related risks of “tearing” of the caliper, while still preserving good sensitivity. Stainless steel is also expected to ensure the necessary robustness and chemical stability, at least over the mission time. In a variation of the embodiment, the flexible sensor arms are formed entirely out of plastic.

FIG. 4 also shows a central connecting point at which all four arms come together. The connecting point is preferably a continuous component that provides structural support for the flexible arms. The connecting point represents, at its maximum width, a distance that is representative of 1.5×, preferably 2×, or preferably 3× the width of the flexible arm at a point most distant from the connecting point. In embodiments, the flexible arms should be considered as being cut from a plane representing a shape similar to a cone with varying slope. Preferably this cone is a secant parabolic cone although in embodiments a tangential parabolic cone can be included. The cone plane represents the four arms of the pig device traveling down a pipeline. The central connecting point serves different purposes for the front and rear set of calipers (FIG. 2d).

In this embodiment, the rear flexible caliper arms may be mounted at the center point of the parabolic cone with the flexible arms advancing in behind the stern section of the pig device. The center point of the flexible caliper arm assembly is connected at the apex or maxima of the parabolic cone surface of the body of the smart pig. The center point may be directly connected to the pig body, such as directly connected at the maximum of the body when it is in a cone-type shape. The central connection point serves as a connectivity hub between the sensor module and the insulated PCB disk 6 of FIG. 2a. The wires 25 in FIG. 6 pass through the support element 22 of FIG. 4 and report the changes in the Ohmic resistance of the interdigitated conductors 24. The wires 25 are directly welded to the PCB disk 7 in FIG. 2a, and the entire caliper-sensors-PCB module is detachable from the foam part. FIG. 5 shows the cylindric extension of the sensor PCB assembly compatible with the central element 13 of FIG. 2A.

For the front set of calipers (which can be further separated from the foam vector 4 by a frontal extension of the central body 13), the flexible arms preferably do not make contact with the body of the pig device. The cylindrical or conical fitting 31 (FIG. 4) connects the hollow central body 13 (FIG. 2C, 2D) with the frontal caliper set. The fitting 31 may be completely submerged in the hollow space 13, or provide some frontal extension further separating the flexible caliper arms and the foam vector. With this separation, the sensors can experience a greater range of deformations without touching the foam vector than in the absence of this spacing. The said frontal extension occupies 0.05-0.1 lengths of the entire device, counted from the front tip of the foam vector. The arrangement resembles an umbrella with a short handle extended over the foam body on the front side, where the handle is the stretch of the fitting 31 protruding along the flow direction beyond the fore-most point of the foam body 4. The support element 22 for the frontal caliper set (FIG. 4) is the foremost point of the device, with the shield 29 fastened between 22 and 13 and sealed by the acrylate or epoxide. In an alternative embodiment, the frontal shield 29 is welded or threaded into the element 22 and represents the fore-most position of the device in the flow.

As shown by reference numeral 6 in FIG. 2a, one or more mounting apparatuses or control centers may be mounted above or below the central point of the caliper arm assembly. This component may include the control apparatus that includes the accelerometer, gyroscope and other circuitry that is connected to the flex sensors and other components. Preferably the controller assembly is mounted as the modular component of the pig device. As such, it is easily accessible for downloading from memory.

Clock Module

The clock module is shown as 21 in FIG. 3. In the preferred embodiment, a real-time clock module is integrated with all other devices via a controller. The role of the clock is to provide the absolute time measurement for the movement with known velocity and accelerations. Knowing these parameters allows one way to compute the longitudinal coordinate of the assembly in the pipe, irrespective to the availability of the GPS back-up. The starting position of the device is recorded automatically since it is injected through a known entry station which is provided with a GPS address, the time of injection is recorded resulting in the complete characterization of the entry point in time and space.

A real-time clock 21 (FIG. 3) or RTC is a computer clock (most often in the form of an integrated circuit) that keeps track of the current time. Although the term often refers to the devices in personal computers, servers and embedded systems, RTCs are present in almost any electronic device which needs to keep accurate time, such as ILI pipe inspection robots. RTC has benefits of low power consumption, a critical requirement for the device that depends on battery life for functioning and needs to spend energy producing multiple records. Most RTCs use a crystal oscillator. The crystal frequency is usually 32.768 kHz the same frequency used in quartz clocks and watches. At 215 cycles per second, it is a convenient rate to use with simple binary counter circuits.

Controller Module

The processor is the main integrating element of the device and function in analyzing, synchronizing and adjusting incoming sensor data. A microprocessor is a computer processor that incorporates the functions of a central processing unit on a single integrated circuit. The microprocessor accepts binary data as input, processes it according to instructions stored in its memory and provides results (also in binary form) as output. Microprocessors contain both combinational logic and sequential digital logic. Microprocessors operate on numbers and symbols represented in the binary number system. Microprocessors can be selected for differing applications based on their word size, which is a measure of their complexity.

In a preferred embodiment, the microprocessor (CPU) supports a micro-controller of a robotic device. Microcontrollers differ from microprocessors per se being a combination of the latter with a set of peripheral functions such as RAM (random access memory), ROM (random operative memory), I/O (input/output functions). In a non-limiting preferred embodiment, the microcontroller is a system-on-a-chip (SOC).

A system on chip is an integrated circuit (also known as a “chip”) that integrates all components of a computer or other electronic system. These components always include a central processing unit (CPU), memory, input/output ports and secondary storage—all on a single substrate or microchip, the size of a coin. It must contain digital, analog, mixed-signal, and often radio frequency signal processing functions, otherwise, it is considered as an “Application Processor”. As they are integrated on a single substrate, SoCs consume much less power and take up much less area than multi-chip designs with equivalent functionality. Because of this, SoCs are very common in mobile computing (such as in smartphones) and edge computing markets. Systems-on-chip are typically fabricated using metal-oxide-semiconductor (MOS) technology.

Systems on Chip contrast the common traditional motherboard-based PC architecture, which separates components based on function and connects them through a central interfacing circuit board. Whereas a motherboard houses and connects detachable or replaceable components, SoCs integrate all of these components into a single integrated circuit, as if all these functions were built into the motherboard. An SoC will typically integrate a CPU, graphics and memory interfaces, hard-disk and USB connectivity, random-access and read-only memories and secondary storage on a single circuit die, whereas a motherboard would connect these modules as discrete components or expansion cards.

In a preferred non-limiting embodiment, the microcontroller is the Arduino platform. Arduino is a prototype platform (open-source) based on an easy-to-use hardware and software. It consists of a circuit board, which can be programed (referred to as a microcontroller) and a ready-made software called Arduino IDE (Integrated Development Environment), which is used to write and upload the computer code to the physical board.

In a preferred embodiment, the microprocessor of the “pig” device includes instructions for an artificial intelligence algorithm). In a non-limiting embodiment, the artificial intelligence processor is a self-programming processor.

Inertial Measurement Unit (IMU).

The Inertial Measurement Unit is a tool allowing navigation at the minimal external positional information (i.e. GPS). A GPS-free navigation is preferred for meaningful interpretation of the ILI device logging which must have a precise positional address for every data point. Building a chain of GPS-receiving stations (“magloggers”) along the path of the pipeline is expensive and does not provide a perfect solution due to the GPS error (see below). The inertial navigation system exists either as a stand-alone or as a supporting tool to the GPS.

An inertial navigation system (INS) is a navigation device that uses a computer, motion sensors (accelerometers) and rotation sensors (gyroscopes) to continuously calculate by dead reckoning the position (in navigation, dead reckoning is the process of calculating one's current position by using a previously determined position, or fix, and advancing that position based upon known or estimated speeds over elapsed time and course without the need for external references).

Conceptually, an accelerometer behaves as a damped mass on a spring. When the accelerometer experiences an acceleration, the mass is displaced to the point that the spring accelerate the mass at the same rate as the casing. The displacement is then measured to give the acceleration. In commercial devices, piezoelectric, piezoresistive and capacitive components are commonly used to convert the mechanical motion into an electrical signal. Piezoelectric accelerometers rely on piezoceramics (e.g. lead zirconate titanate) or single crystals (e.g. quartz, tourmaline). The piezo-materials are advantageous in terms of their upper-frequency range, low packaged weight, and high-temperature range. Piezoresistive accelerometers are preferred in high shock applications. Capacitive accelerometers typically use a silicon micro-machined sensing element. Their performance is superior in the low-frequency range and they can be operated in servo mode to achieve high stability and linearity.

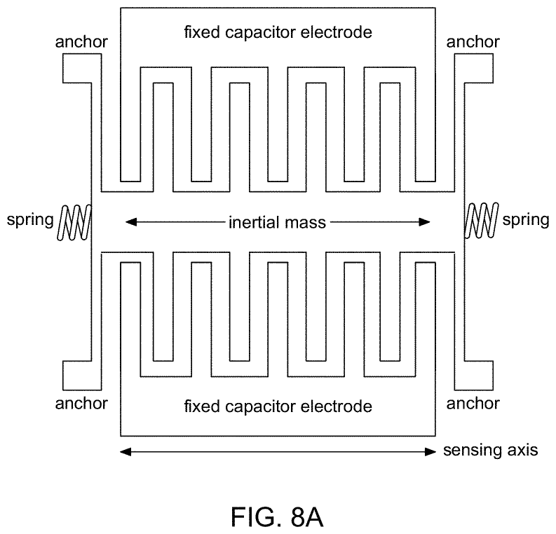

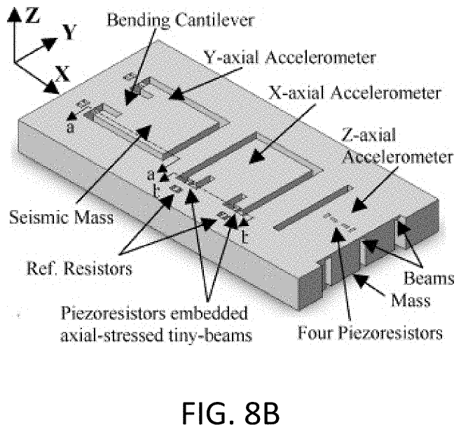

The accelerometers is preferably a micro-electro-mechanical system (MEMS) consisting of a cantilever beam with a proof mass (also known as seismic mass). Damping results from the residual gas sealed in the device. As long as the Q-factor is not too low, damping does not result in a lower sensitivity (Q factor is a dimensionless parameter that describes how underdamped an oscillator or resonator is. It is defined as the ratio of the peak energy stored in the resonator in a cycle of oscillation to the energy lost per radian of the cycle). FIGS. 8a and 8b illustrate the typical schemes for MEMS accelerometers, suitable for the IMU of the disclosure.

FIG. 8A presents a uniaxial capacitive micro-accelerometer. The capacitance of the space between the capacitor electrodes depends on the proximity and symmetry of the positions of the interdigitated extensions originating in the inertial mass and in the capacitance electrodes. The extensions on the side of the electrodes are metallic, while the extensions on the side of the inertial mass element are dielectric. The dielectric constant of this material differs from that of vacuum or air, and therefore the positional shift of the dielectric “fingers” vs. the metallic electrode extensions produces a voltage change, proportional to the magnitude of acceleration. The extent of displacement of the inertial mass is regulated by a pair of the springs, responding in the elasticity range by the Hooke's Law.

FIG. 8B presents a triaxial piezoresistive micro-accelerometer. The key element in this device are piezo bars, experiencing contraction or extension in proportion to the extent of acceleration. The movement of the seismic mass element deforms the bars that produce a proportional signal based on either the piezoelectric effect (re-alignment of inherent crystal cell dipoles under mechanical stress) or due to the piezo-resistive effect (change in ohmic resistance of a semiconductor material in proportion to mechanical forces). The arrangement of the device detects accelerations in all three dimensions. Most micromechanical accelerometers operate in-plane, that is, they are designed to be sensitive only to a direction in the plane of the die. By integrating two devices perpendicularly on a single die a two-axis accelerometer can be made. By adding another out-of-plane device, three axes can be measured (FIG. 8b). Such a combination may have much lower misalignment error than three discrete models combined after packaging.

Another type of MEMS-based accelerometer is a thermal (or convective) accelerometer that contains a small heater at the bottom of a very small dome, which heats the air/fluid inside the dome producing a thermal bubble that acts as the proof mass. An accompanying temperature sensor (like thermistor; or thermopile) in the dome is used to determine the temperature profile inside the dome, hence, letting us know the location of the heated bubble within the dome. Due to any applied acceleration there occurs a physical displacement of the thermal bubble and it gets deflected off its center position within the dome. Measuring this displacement the acceleration applied to the sensor can be measured. Due to the absence of solid proof mass, thermal accelerometers provide high shock survival.

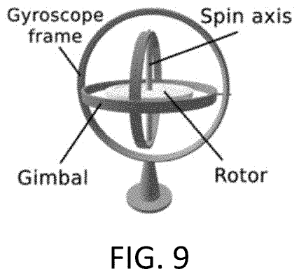

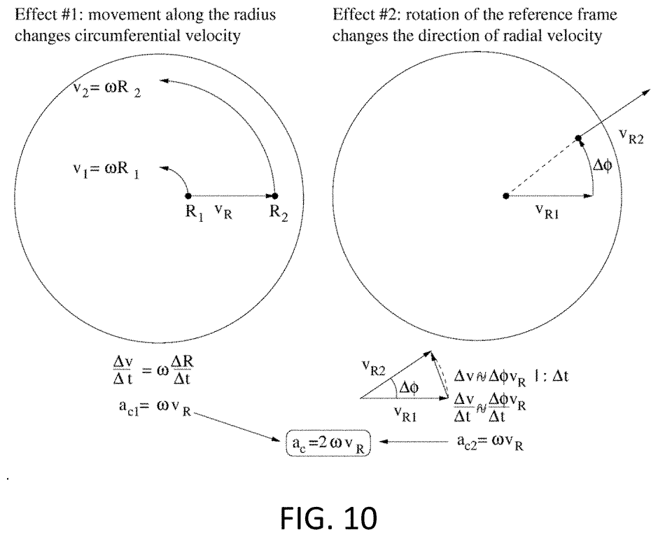

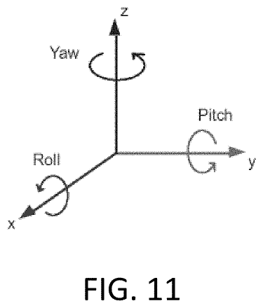

A gyroscope is a device used for measuring or maintaining orientation and angular velocity. In its original rotational embodiment, it is a spinning wheel or disc in which the axis of rotation (spin axis) is stabilized by conservation of angular momentum (FIG. 9). When rotating, the orientation of this axis is unaffected by tilting or rotation of the mounting (gimbals and frame), according to the conservation of angular momentum. The rotation of the mountings follows the inertial forces that develop upon rotating the system, and unlike the wheel, these components follow the inertial forces. The angles between the rotation axis and the mountings provide the information about the turns and the direction of movement vs. the original direction, when the rotational energy was imparted to the disk. The gyroscope of FIG. 9 is unsuitable for the disclosed method.

Some systems incorporate multiple gyroscopes and accelerometers (or multiple-axis gyroscopes and accelerometers), to achieve output that has six full degrees of freedom. These units are called inertial measurement units, or IMUs.

In a preferred non-limiting embodiment, the accelerometer and gyroscope are interconnected as parts of an Inertial Mapping Unit (IMU) (See: Li R, Cai M, Shi Y, Feng Q, Chen P. Technologies and application of pipeline centerline and bending strain of In-line inspection based on inertial navigation.

Integration of accelerometer and gyroscope with other devices—such as the caliper arm deformation discussed earlier further increases the robustness of information-gathering and allows each individual component to remain reliable, simple and inexpensive. For example, an indication of a pipeline contraction by a caliper arm is accompanied by an IMU input by both accelerometer and gyroscope components, since the position of the “pig” device is expected to be shifted relative the hydrocarbon flow, it experiences axial rotations and linear acceleration due to flow acceleration in the contraction section. Similar effects—but of the opposite sign—are observed for the expansions in pipelines due to the manufacturing defects or loss of metal. Over multiple situations, the caliper and IMU would produce correlated signals, and therefore the absence of a signal by one component (a caliper arm missed a defect on a pipe wall, as a non-limiting illustration) is compensated by an IMU signal of the same pattern when the caliper component is present. In this respect, the inventive system utilizes an interrupt detection method such that whenever any sensor gets updated, it records the corresponding value along with all other sensors' values, whether updated or not, and produces the time stamp of that instance to the SD (memory) card.

Odometer

The odometer may be electronic, mechanical, or a combination of the two (electromechanical). Most odometers work by counting wheel rotations and assume that the distance traveled is the number of wheel rotations times the circumference. A wheel can be installed within the “smart pig” apparatus and actuated by the displacement. In an embodiment, the odometer comprises a slotted wheel and an opto-interrupter assembly. Whenever a slot is detected by an opto-interrupter, an interrupt is generated which leads to the recording of the current displacement. Any newly detected displacement is added to the previous displacement value/s to give the distance covered. A distance range of >10⁶ meters can be achieved without fatal errors.

The presence of an odometer facilitates the data fusion necessary for achieving the precise positional address of the suspected defects in the pipe and provides a means for confirming calculated distance values. The direct measurement of the pig's position may interact with the accelerometer position model

Power Source

Other components of the system may be a re-chargeable battery and a battery holder.

The non-limiting examples for the power sources suitable for the ILI “smart pig” devices are lithium ion batteries with or without supercapacitor equalizers.

Optional Sensors

In some embodiments, MFL (magnetic flux sensors) are included as an additional sensor in the device. Magnetic flux leakage (TFI or Transverse Field Inspection technology) is a magnetic method of nondestructive testing that is used to detect corrosion and pitting in steel structures, most commonly pipelines and storage tanks. The basic principle is that a powerful magnet is used to magnetize the steel. At areas where there is corrosion or missing metal, the magnetic field “leaks” from the steel. In an MFL (or Magnetic Flux Leakage) tool, a magnetic detector is placed between the poles of the magnet to detect the leakage field. The chart recording is interpreted to identify a leakage field that corresponds to damaged areas and provides a basis for an estimate of the depth of metal loss.

In other embodiments, defects are sensed by an EMAT ultrasonic transducer (UT) with piezoelectric assistance. An electromagnetic acoustic transducer (EMAT) is a transducer for non-contact acoustic wave generation and reception in conducting materials. Its effect is based on electromagnetic mechanisms, which do not need direct coupling with the surface of the material. EMATs is a process that comprises a magnetic field B directed normally to a metal surface. In the direction of the magnetic vector, the second emitter radiates an alternating electromagnetic field with the frequency f. The field reaches the metal and polarizes the conducting electrons such that the field is compensated beyond a very thin “skin deep” layer (Faraday caging effect). In the skin-deep layer, the alternating magnetic flux (AC) produces the perpendicular circular eddy currents (according to the fourth Faraday's law), coplanar with the metal surface. These eddy currents are also perpendicular to the magnetic vector B, and therefore the current-carrying elements experience the mechanical Lorentz force, proportional to the product of the magnetic flux time derivative (dAC/dt) by the constant magnetic field B. The resulting tangential oscillations of the metallic surface produce ultrasound. If the surface is cracked, the acoustic signature deviates from the signature of an intact surface. Due to this coupling-free feature, EMATs are particularly useful in harsh, i.e., hot, cold, clean, or dry environments. EMATs are suitable to generate all kinds of waves in metallic and/or magnetostrictive materials. Depending on the design and orientation of coils and magnets, shear horizontal (SH) bulk wave mode (norm-beam or angle-beam), surface wave, plate waves such as SH and Lamb waves can be excited for nondestructive testing (NDT) of metallic structures (See: Klann M, Beuker T. Pipeline Inspection With the High Resolution EMAT ILI-Tool: Report on Full-Scale Testing and Field Trials. In 2006 International Pipeline Conference 2006 Jan. 1 (pp. 235-241). American Society of Mechanical Engineers Digital Collection; Willems H, Jaskolla B, Sickinger T, Barbian A, Niese F. A new ILI tool for metal loss inspection of gas pipelines using a combination of ultrasound, eddy current and MFL. In 2010 8th International Pipeline Conference 2010 Jan. 1 (pp. 557-564). American Society of Mechanical Engineers Digital Collection; U.S. Pat. Nos. 7,657,403; 8,319,494; incorporated herein by reference in entirety).

Pulsed-eddy current (PEC) tools use a probe coil to send a pulsed magnetic field into a metal object. The varying magnetic field induces eddy currents on the metal surface. The tool processes the detected eddy current signal and compares it to a reference signal set before the tool run; the material properties are eliminated to give a reading for the average wall thickness within the area covered by the magnetic field. The tool logs the signal for later analysis (See: Niese F, Yashan A, Willems H. Wall thickness measurement sensor for pipeline inspection using EMAT technology in combination with pulsed eddy current and MFL. In 9th European Conference on NDT, Berlin 2006 September (Vol. 18, pp. 45-52); Willems H, Jaskolla B, Sickinger T, Barbian A, Niese F. A new ILI tool for metal loss inspection of gas pipelines using a combination of ultrasound, eddy current and MFL. In 2010 8th International Pipeline Conference 2010 Jan. 1 (pp. 557-564). American Society of Mechanical Engineers Digital Collection; Mazraeh A A, Ismail F B, Alta'ee A F. RFEC PIG designed for long distance inspection. In IPTC 2014: International Petroleum Technology Conference 2014 Jan. 19 (Vol. 2014, No. 1, pp. cp-395). European Association of Geoscientists & Engineers; incorporated herein by reference in entirety).

Laser profilometers project a shape onto an object surface. Superficial anomalies (e.g., pitting corrosion, dents) distort the shape, allowing the inspection technicians to measure the anomalies using proprietary software programs. Photographs of these laser distortions provide visual evidence that improves the data analysis process and contributes to structural integrity efforts. Laser profilometry is suitable in gas lines, but not for bulke petroleum liquids.

In an alternative embodiment, acoustic resonance technology (ART) is an acoustic inspection technology. ART exploits the phenomenon of half-wave resonance, whereby a suitably excited resonant target (such as a pipeline wall) exhibits longitudinal resonances at certain frequencies characteristic of the target's thickness. Knowing the speed of sound in the target material, the half-wave resonant frequencies can be used to calculate the target's thickness. In a closely related technique, the presence of cracks in a solid structure can be detected by looking for differences in resonance frequency, bandwidth and resonance amplitude compared to a nominally identical but non-cracked structure. The method was able to detect mm-size cracks in as-cut and processed silicon wafers, as well as finished solar cells, with a total test time of under 2 seconds per wafer.

Launching of the Device

The robotic pipeline inspection device (smart pig) is designed so that the pig is loaded into a launcher, which is pressured to launch the pig into the pipeline through a kicker line. In some embodiments, the pig is removed from the pipeline via the receiver at the end of each run. The system allows for the receipt and extraction of pigs at the launcher, as blockages in the pipeline may require the pigs to be pushed back to the launcher.

The pig is pushed either with a gas or a liquid; if pushed by gas. The device of the present disclosure is preferably for pigging liquid flow pipelines.

In general, the valves are provided that isolate the entry port of the pig device from the pressure, the port is evacuated to a safe pressure level, the pig is inserted and after closing the port the connecting valve to the main pipeline is re-opened.

GPS-Free Navigation

Inertial navigation systems tend to suffer from integration drift: small errors in the measurement of acceleration and angular velocity are integrated into progressively larger errors in velocity, which are compounded into still greater errors in position. Since the new position is calculated from the previous calculated position and the measured acceleration and angular velocity, these errors accumulate roughly proportionally to the time since the initial position was input. Therefore, the position is preferably be periodically corrected by input from some other type of navigation system.

Accordingly, inertial navigation is preferably used to supplement other navigation systems, providing a higher degree of accuracy than is possible with the use of any single system. For example, if, in terrestrial use, the inertially tracked velocity is intermittently updated to zero by stopping, the position will remain precise for a much longer time, a so-called zero velocity update.

Estimation theory in general and Kalman filtering in particular, provide a theoretical framework for combining information from various sensors. By properly combining the information from an INS and other systems (GPS/INS), the errors in position and velocity are stable.

One object of the present disclosure is to provide a GPS-free navigation system with minimal error. Such an objective is realistic when positional estimates are periodically corrected by reference benchmarks.

A non-limiting example of reference benchmarks (reference points) are welding seams. The welding seams are visible by the magnetic flux, inductive and acoustic methods of analysis, due to the differences in the structure and chemical composition of the original pipe metal and the welding seam. The ultrasonic principle is also applicable for the same. Welding seams collide with the odometer's wheels and are detectable by the caliper arms, producing a pattern of elevation on one side and depression on another. The transition from the previous caliper position to the new position is sharp, producing high values of the first derivative of caliper arm displacement per positional change. The presence of a welding seam mismatch would produce a change in the fluid flow; therefore the “pig” device experiences a lateral shift (acceleration) and a rotational component, both are detectable by the IMU module as described above.

Each welding joint creates a new reference point, and the positional errors that accrue between the joints are summarized and classified, to ensure that the real reading accounts for the errors by a correction protocol. The correction protocol introduces the contexts in which errors develop, such as: elevation, descent, contraction, expansion, corrosion, turns, colder and warmer stretches, less loaded and more loaded stretches, valley or mountain regions, softness or hardness of ground without limiting. In each individual category, and in a large but finite number of category combinations the errors tend to group. There is no need to completely nullify the positional error, since the estimated problematic location can be investigated more rigorously by a more comprehensive ILI tool, and on a limited trajectory the magloggers or pig detection sensors can be deployed. Thus, the positional error should not become prohibitively high and preferably should not exceed a single pipe segment.

In another non-limiting embodiment, the direction of ascent or descent of the device is detectable by the IMU and odometer working in combination. Before the pipe segment turns upwards, the flow pattern changes according to the flow continuity and Bernoulli laws, and a sharp signal (roll and/or pitch) is detected by the IMU module, allowing to establish the ascent angle. The length of the ascent is determined by the odometer, until the combination of detected turns points to reaching a flat peak. The altitude of the peak (vs. the sea level) compares with the altitude used in the construction of the pipeline, with the plan loaded in the processor memory of the robotic device. Thus, the coordinate of the local peak is identified, and this elevation serves as an additional external benchmark for the positional error resets. Analogously, the IMU detects any other turns and interprets this information in alignment with the pipeline construction plan generating the positional error resets.

In another embodiment, applicable to shorter stretches of several hundred meters between the pig launching and receiving stations, the logging data are compared with the real distance between the entry and exit ports, and the error is evenly attributed to each pipe segment. This is possible and acceptable in the assumption that the error predominantly accrues due to numerical error of integration. The trend of accrual may be non-linear with the distance, can be studied and accordingly apportioned between the individual segments.

In a preferred embodiment, all methods of the positional error reset are applied concordantly. Ideally, only welding joint mismatches should be suitable to produce the corrective reference data, such defects follow the Poisson's distribution and in many cases are below the sensitivity threshold of caliper and IMU sensors. All joints can be detected by MFL and acoustically, but these methods are demanding on the power supply, and the battery life is short. This disclosure focuses on the non-optional minimal set of sensors (IMU, calipers, odometer). Thus, only a random subset of pipe welding joints contributes the error reset points and needs to be supported by other components.

In the most preferred embodiment, the “pig” device operates under the control of a processor capable of artificial intelligence self-programming, which implements the position error self-correction. The processor includes instructions to estimate and subtract the positional error for a stretch of path originating in the last reference point with a known position (where the error was last nullified). The processor is trainable by comparing the estimated positions by the “pig” devoice sensors and comparing them with the real positions in the reference points. At each comparison, the self-training algorithm continues to improve its predicting ability by including the new comparison in a training set and continually “learning” on its errors.

The processor considers a first plurality of factors that correlate with positional errors. Without limiting, such a list comprises: the diameter of the pipe, the diameter of the pig device, the distance between the pipe wall and the outer diameter of the device, the buoyancy of the foam vector in the hydrocarbon flow, the steepness of ascents and/or descents, the number of turns, the number of degrees of turning per a covered mile of trajectory, viscosity of the fluid, the presence of aggregates in the fluid flow, the temperature of the fluid, the presence of contractions and expansions in the pipeline, the ratio of the pig's length to diameter, the ratio of the radius of the frontal (face) side of the device to its length and/or to the diameter of the cylindrical part of the foam vector, compressibility of the foam, the number of caliper arms in contact with the wall, the mass of the device, the smoothness of the device's outer surface, mass distribution between the front and rear ends of the device, the presence or absence of a rigid skeleton tube, the size of the disk, the rigidity of the caliper arms, the pressure in the pipeline, the average linear velocity in the pipeline, the volume flow per second (m3/sec), mass flow per second (kg/sec), the Reynolds number (Re) in the pipeline, the profile of local velocity distribution along the radius of the flow cross-section (parabolic for a purely laminar flow with Re<2100), density of the fluid, asphaltene content of the fluid, the percent of dissolved gas in the fluid, smoothness of the pipeline walls, the presence of deposits on the walls, the mileage on the “pig” device, the distance since the last reference point (the error can accumulate disproportionally over longer distances), gradually increasing acceleration, randomly varying acceleration, but increasing the moving average, leaving the moving average constant, but introducing random spikes in acceleration, the cycles of acceleration and deceleration of equal length and intensity, of unequal length and intensity, interval function of acceleration (spike and stop), profiled variation of acceleration, the ratios, the products, the sums, any mathematical functions of any of the members of the list.

All primary values of the factors are known for the device and the pipeline, and the software selects the values that best correlate with the positional error. In a non-limiting embodiment, the software uses linear regression to minimize the differences between the predicted values of the errors based on the features above and the real values of the errors, determined by comparing with the positional references.

A second plurality comprises a list of mathematical forms combining the first plurality of factors. The list of error-related factors in a pipeline can be reduced due to negligible impacts for most of them. The reduced list of factors can be combined in a linear combination, each factor included with a training (floating) coefficient, the latter to be determined in the least square method procedure.

In another embodiment, the mathematical form is the product of each factor raised to power, the power coefficients are floating and are established by the least square method. In yet another embodiment, each factor's contribution is a polynomial with n members, or a combination of exponentials or trigonometric functions. The mathematical forms above are only non-limiting examples and do not represent the entire range of possibilities.

The software begins with recording from 20 to 30 runs of the device between the known reference points. For example, the device starts at the launch station and passes 30 welding seams. On each stretch, the IMU computes an estimate of a distance and the real distance is known from the pipeline plan. The errors are recorded for each 20-30 segments. The first plurality of factors is correlated to the measured errors and the most correlating 4-5 factors are included in the first “green” model, preferably written as a linear combination of factors taken with the floating weight coefficients. The weights are determined by linear regression, minimizing the discrepancy between the weighted combinations of factors and the measured errors for each segment 1-30.

If the initial list of training segments is 30, the reference point 31 is outside of the training set. The prediction rule developed earlier is applied now and the discrepancy between the IMU data and real position is determined for 31. If this discrepancy is the same as expected based on the linear combination of factors, the point 31 is defined as successful, and the choice of factors predicting positional errors remains the same. Assuming, for sake of argument, that the reference point 32 shows a mismatch between the IMU distance and real distance that strongly disagrees with the factor model. The criterion of a breakdown is when the predicted error is by at least 50% different from the observed positional error.

This event triggers the inclusion of the break-down point 32 in the training set and re-training of the entire procedure by selecting new best correlating factors and new weight coefficients. Of note, each point where the model breaks down is weighted more heavily for the inclusion in the new training set by introducing the emphasis coefficient. The emphasis coefficient is 5-6 for 30 training points, 50-60 for 300, 500-600 for 3000, and comprises the number of times the break-down point data are included in the new training set. For example, the old training set of 30 points is extended by 6 identical points, each is a copy of the breakdown point. If there is no breakdown, the training set is still expanded, but by just one value of the agreement point. For example, the old training set of 30 points is extended by only 1 agreement point.

In this approach, the model adapts to recognize more and more diverse new situations, and this adaptation is achieved by a continuous, evolution-like selection of the best predictors out of the practical infinity of possibilities. As the model continues to expand and encompass more and more points that the device covered during its motion in the pipeline, the set of factors that correlates with the progressing model changes. The initially optimal factors lose significance and the new factors or combinations become more prominent. Eventually, the fully evolved model takes into account all diversity of local conditions in the pipeline and therefore the breakdowns become exceedingly rare.

In an embodiment, to make the training process more intense, the “pig” device includes the magnetic flux, inductive and sonic sensors capable of detecting each welding joint, providing more positional references and accelerating the training process. Once the process is complete, the data is uploaded on a simpler “pig” device without the expensive sensors and relying only on the calipers, IMU, odometer and timer.

Eventually, the final product of this selection process is a universal algorithm that satisfactorily predicts the positional errors under most of possible circumstances. The criterion of satisfactory prediction is when the defect identified as present in the pipe segment N is indeed within that segment. The properly trained program stops self-training at this point. It predicts positional errors correctly without positional referencing. In a less ideal scenario, the pipeline inspection device still needs some external referencing, but can utilize the cost-effective referencing available through the big turns, ascents and descents or the addresses of the receiving stations. The reliance on the costly welding joint detection becomes minimal.

Diagnostics of Defects in a Pipeline

The addition of the features supporting the predictive modeling (diverse sensors, including MFL, EMAT and acoustic), enables the analyst operating the device with the opportunity to prioritize the problematic sites. The addition of multiple detection modalities makes the device more sensitive and discriminative between the natural features of the pipeline and the accrued or growing defects. At the same time, such apparatuses are more expensive, more demanding in terms of battery life and require professional supply chains, as opposed to being produced from locally available components. The invention disclosure further concerns with the methods of use, wherein the sensor set is defined as minimal (IMU, odometer, calipers, timer) or optionally complete/comprehensive (added MFL, EMAT, acoustic components).

Internal corrosion in pipelines is often caused by water, sediment, or chemical contaminants present in the multi-phase flow. This normally occurs at the bottom of the pipe and at low points in the pipeline where sediment and water can settle out of the product being transported, therefore creating narrow and long defects.

In one non-limiting embodiment, the effect of corrosion defects on the collapse pressure of pipelines is predictable by simple caliper-only measurements of corrosive wall ovalization, inherently linked to structural weakening. In another non-limiting embodiment, caliper-only tool detects deformations of the pipe in the areas of supports which were caused by washouts.

While in the above-mentioned embodiments a stand-alone caliper can diagnose the largest and the most obvious defects, smaller defects can be also uncovered by data-fusion available in the minimal assembly comprising a timer, odometer, gyroscope, accelerometer and flexural electronic caliper sensors. The improved detection accounts for the translational and rotational accelerations sensed by the combination of the devices. Together, the results by the IMU, odometer and calipers form a signature that can be recognized by an artificial intelligence algorithm and attributed to a class.

The classes comprise, for example:

no defect situation;

original defect of pipeline construction (mismatch during welding);

corrosion- or deformation-caused lesions.

In the preferred embodiment, an artificial intelligence algorithm is trained to recognize each class of the data based on the signatures in the acceleration and caliper datasets.

The physical basis for the resolution between the non-defect status and defect status, as well as between the original defects and the novel, developing situations is the balance between the friction and inertial forces of the flow that carries the intelligent caliper. In a non-limiting example, a wash-out situation means a long groove or pit in the pipe wall on one side and the absence of the same on the other. The asymmetry is detected by the flexural sensors and produces a lateral acceleration component in the flow (and in the motion of the pig caliper) toward the damaged wall. If the wash-out is extensive, it increases the cross-section and causes the translational deceleration of the flow.

In another embodiment, the defect is rust and the corrosion products build-up in a section of a pipe. The roughening of the contact surface on the pipe creates turbulence and a local drop of hydrostatic pressure. The local velocity profile in the fluid becomes sharper (it is either parabolic in a strictly laminar flow over an ideally smooth wall, or distorted parabolic when the Reynolds number increases >2300). The change of the hydrodynamic regime alters the Yaw, Pitch and Roll readings of the gyroscope and of the translational accelerometer. The corrosion sites produce time-variable, randomized and asymmetric signal by the flexural caliper sensor and IMU.

In another non-limiting embodiment, a welding mismatch is revealed as two matching defects of opposite sign detected by the counterposing caliper arms and as a side-way acceleration shift detected by the IMU unit, as described above. The cracks—unless very wide—do not produce the acceleration signature and do not interact with the flexural caliper sensor; therefore the additional optional sensors described above are relevant. High-resolution MFL tools collect data approximately every 2 mm along the axis of a pipe and this superior resolution allows for a comprehensive analysis of collected signals. Pipeline Integrity Management programs have specific intervals for inspecting pipeline segments and by employing high-resolution MFL tools corrosion growth analysis can be conducted. This type of analysis proves useful in forecasting the inspection intervals. Although primarily used to detect corrosion, MFL tools can also be used to detect features that they were not originally designed to identify. When an MFL tool encounters a geometric deformity such as a dent, wrinkle or buckle, a very distinct signal is created due to the plastic deformation of the pipe wall. In MFL data, a dent is easily recognizable by trademark “horseshoe” signal in the radial component of the vector field. What is not easily identifiable to an MFL tool is the signature left by a crack. A purely acoustic method, such as ultrasonic detection is also applicable to crack detection.

The presence of significant lesions in the metal in multiple locations or the presence of multiple bulges and deformations indicates a high-risk installation in need of more detailed analysis, available through the data fusion of the timed caliper, IMU, MFL, EMAT and ultrasonic signals. In contrast, a relatively “clean” profile by the disclosed method points to a lower probability of the fatal defects. These longitudinal patterns of the suspected defects obtained by a cheaper tool can be catalogued and compared with the benchmark tests by a more expensive integrated tool, detecting all classes of damage.

In the preferred embodiment, these patterns are analyzed by an artificial intelligence software analogous to the listed above or based on the different principles without limitation. The software is trained to recognize the original defects and the developing defects and propose a score to the defects, based on the industry experience. The software can be trained by producing a training set of pipe segments incorporating the typical defects and applying a self-programming processor to minimize the false negatives and false positives, based on the algorithmic principles described above or on different principles, without limitation.

Alternatively, the modular structure of the apparatus and the standardization of the PCB/sensor unit and the controller allows collecting a library of signatures, different for the pipelines of various diameters, velocities, working pressures. This library of prior experience is re-analyzed by the artificial intelligence software, and the common features of the defects are extracted across variations in conditions.

In a preferred embodiment, the artificial intelligence examines preliminary screening data by the inexpensive inventive foam caliper pig tool (with the minimal sensor set), collected over many kilometers of the pipeline and takes a decision to send a more expensive “smart pig” device to the regions that show more problematic preliminary patterns. This more expensive tool utilizes fewer capabilities in the safer regions and relies on more data sources in the more damaged regions. Thus, the overall inspection becomes cheaper and more streamlined, enabling processing greater lengths of the piping, combining a greater process speed and the comparable standard level inspection quality.

The defects described up to this point can be static (welding mismatches, bent segments, originally present dents in the pipe walls) and developing (corrosion, strain, deposits, washouts, deformations). The second category is more problematic since the pipeline is functioning with static defects but may reach a critical state with the combination of dynamic damaging factors. The defects are catalogued and correlate with the recognizable patterns in each diagnostic method. The indication of the same type of defects by multiple methods reinforces the diagnosis. Certain irregularities (cracks, corrosion) tend to propagate in time, and their size, depth and pattern within the context of the transported and piping materials is predictive of the future size and the extent of risk.

In some non-limiting embodiments, the prediction of future pipeline integrity and safety incorporates a single ILI run, providing multiple signatures of damage through various sensing methods. The signatures are compared with the prior accumulated knowledge base of similar patterns and of the outcome correlates. In other non-limiting embodiments, the estimates are conducted based on time course of damage development assessed by the repeated ILI launches.

In the preferred embodiment, the inexpensive and long-range inventive ILI method is calibrated by a more expensive multi-sensor method establishing a correlation between the caliper and IMU signature of the inventive method and the conclusions of the expensive benchmark method. Then, the inventive foam “pig” device can be repeatedly launched over many kilometers of the pipeline to collect the time-dependent information about the state of the line. The sections demonstrating the fastest rate of signature drift are likely to be the most dangerous in terms of possible breakdown and need to become the focus of a more detailed study by more expensive ILI tools and possibly—disconnection and replacement,

Having generally described this disclosure, a further understanding can be obtained by reference to certain specific examples which are provided herein for purposes of illustration only and are not intended to be limiting unless otherwise specified.

1. A robotic pipeline inspection device for inspecting a pipeline containing a hydrocarbon fluid, comprising:
an accelerometer and a gyroscope, combined in an Inertial Mapping Unit (IMU);
an odometer;
a real-time clock module;
a system controller;
a memory card;
a removable and rechargeable battery;
a foam vector;
wherein said foam vector is shaped as a parabolic cone-shaped cylinder;
wherein the foam vector comprises an axial hollow support member traversing an entire length of the foam vector;
wherein the system controller, the real-time clock module, the IMU, the odometer, and the memory card are assembled on a printed circuit board (PCB) and disposed in a compartment impermeable to the hydrocarbon fluid in the pipeline;
wherein a flat flange connects the foam vector to said compartment;
wherein the compartment is disposed within the foam vector;
wherein the robotic pipeline inspection device further comprises at least two flexible arm electronic calipers and at least two flexural caliper sensors configured to measure an inner diameter of the pipeline;
wherein the flexible arm electronic calipers are mounted at a center point of the parabolic cone-shaped cylinder by a plurality of plastics supports disposed on the flat flange and configured to trail a stern section of the robotic pipeline inspection device when operated in the pipeline;
wherein the robotic pipeline inspection device is assembled in the order from a bow section to the stern section: the foam vector; the flat flange, the compartment; and a central point of said flexible arm electronic calipers;
wherein the flexible arm electronic calipers have caliper sensor wires, one sensor wire per arm caliper, and the sensor wires originate in the compartment and extend to said central point of the flexible arm electronic calipers; and
wherein the device comprises a set of flexible caliper sensors installed at the front of the robotic pipeline inspection device.

2. The device of claim 1, wherein the gyroscope and the accelerometer comprising the IMU are micromechanical MEMS devices.

3. The device of claim 2, wherein the gyroscope is a vibrating structure triaxial gyroscope; wherein the MEMS gyroscope is flat and detects yaw, roll and pitch in a single planar device.

4. The device of claim 2, wherein the accelerometer includes at least one of a capacitive detector and a piezoelectric detector; wherein the accelerometer is flat and detects acceleration in positive and negative directions along X, Y and Z axes.

5. The device of claim 1, wherein the foam vector is made from expanded polyurethane foam having a density 0.03-0.1 g/cc.

6. The device of claim 1, wherein the system controller, the printed circuit board, compartment, the flat flange, and the flexible arm electronic calipers form a single unit detachable from the foam vector.

7. The device of claim 1, wherein the compartment comprises an insulated USB port which is adapted to upload and download shareware, software and datasets.

8. The device of claim 1, wherein said hollow support member houses an MFL sensor, an EMAT sensor and/or an ultrasonic sensor.

9. The device of claim 8, wherein the hollow support further houses at least one of a resonance ultrasonic vibration (RUV) sensor and an acoustic resonance sensor.

10. A method for inspecting a pipeline, comprising
injecting the robotic pipeline inspection device of claim 1 at an entry station into the pipeline,
moving the robotic pipeline inspection device through the pipeline to an exit station,
wherein during the moving, the hydrocarbon fluid is present in the pipeline in a non-compressible pressure range of 5-500 atm.

11. The method of claim 10, further comprising:
recording one or more sections of the pipeline having one or more defects selected from the group consisting of a corrosion, a washout, a bend, a bulge, a welding mismatch, an ovalization, a concavity, a groove, a trough, and a convexity.

12. The method of claim 10, comprising:
identifying a crack in the pipeline using an ultrasonic signature of the crack.

13. The method of claim 12, further comprising:
calibrating the flexural caliper sensors of the robotic pipeline inspection device with at least one of an EMAT analysis, an MFL analysis, an ultrasonic analysis, and an acoustic analysis.

14. The method of claim 13, wherein the calibrating utilizes data from both the flexural caliper sensors and an instrument disposed in the hollow support member.

15. The method of claim 11, further comprising:
establishing a positional address of the robotic pipeline inspection device in the pipeline in a GPS-independent manner.

16. The method of claim 15, wherein the positional address of the robotic pipeline inspection device is established according to welding seams between individual pipe segments of the pipeline.

17. The device of claim 1, wherein the PCB further comprises:
a same amount of variable impedance circuits as an amount of the flexural caliper sensors;
an analog processor; and
a multiplexer,
wherein the system controller is positioned in a center of the PCB and is separately connected to each of the real-time clock module, the IMU, the odometer, the multiplexer, the memory card, and the analog processor and is configured to coordinate recording of data between each component of the PCB,
wherein the memory card is connected to the real-time clock module, the IMU, the odometer, the multiplexer, the memory card, the system controller, the variable impedance circuits, and the analog processor, and is configured to receive and store data from each component of the PCB,
wherein each variable impedance circuit is separately connected to the analog processor which is configured to provide a converted signal of each variable impedance circuit,
wherein the analog processor is connected to and provides the converted signal of each variable impedance circuit separately to the system controller, and
wherein the multiplexer is connected to the real-time clock module, the IMU, the odometer and is configured to provide a single output of all input data to the system controller.

18. The device of claim 17, wherein PCB input wires protrude and pass through pinholes made in a bottom face of the compartment and are connected to the flexural caliper sensors.

19. The device of claim 18, wherein the flexural caliper sensors comprise variable interdigitated resistors configured to vary in resistance with an amount of bending, and
wherein the variable interdigitated resistors are connected to the variable impedance circuits via the PCB input wires to produce a corresponding voltage value.

20. The device of claim 1, further comprising a shutter disposed within the hollow support member in a transversal position.
